(19) United States
(12) Patent Application Publication
(10) Pub. No.: US 2005/0154277 A1
(43) Pub. Date: Jul. 14, 2005

(54) Apparatus and methods of using built-in micro-spectroscopy micro-biosensors and specimen collection system for a wireless capsule in a biological body in vivo

(76) Inventors: Jing Tang, Cambridge, MA (US); Leming Wang, Bayside, NY (US); Jinpin Ying, East Brunswick, NJ (US); Weilong Lee, West Covina, CA (US); Pingpei Ho, New York, NY (US)

Correspondence Address: Ping Ho, #7H, 300 Albany street, New York, NY, 10280, US

(21) Appl. No.: 10/747,005

(22) Filed: Dec. 29, 2003

Related U.S. Application Data
(60) Provisional application No. 60/437,022, filed on Dec. 31, 2002.

(51) Int. Cl. A61B 5/05

(52) U.S. Cl. 600/407

(57) ABSTRACT

A wireless capsule as a disease diagnosis tool in vivo can be introduced into a biological body by a native and/or artificial open, or endoscope, or an injection. The information obtained from a micro-spectrometer, and/or an imaging system, or a micro-biosensor, all of which are built-in a wireless capsule, can be transmitted to the outside of the biological body for medical diagnoses. In addition, a real-time specimen collection device is integrated with the diagnostic system for the in-depth in vitro analysis

### FIELD OF THE INVENTION

The present invention related to an apparatus and method for diagnosing diseases inside of a living biological body. A wireless capsule comprises a micro-spectrometer, a biosensor and/or a select specimen collection system and can be introduced into a nature tract of the biological body. The disease information can be acquired during the wireless capsule travels through the biological body.

### BACKGROUND OF THE INVENTION

Wireless capsule means a micro-device, which can travel inside of a living biological body for collecting information to diagnose diseases and/or collecting specimen. Spectroscopy means a technique of measuring an optical property distribution or a concentration from a biological tissue and/or juice to diagnose disease via its morphology and/or chemical component changes. Biosensor means a self-contained integrated device, which is capable of providing specific analytical information using a biological recognition element.

One of the primary benefits of the photonic approach to imaging and examining biological materials is that said imaging and examination can be conducted in vivo in a patient with little risk of injury to the patient. This is to be contrasted with certain conventional imaging techniques, such as X-ray imaging, which involves subjecting a patient to potentially harmful X-ray radiation, and with certain conventional examination techniques, such as biopsy and histological evaluation, which cannot be conducted in vivo. The organ or tissue to be examined is located internally. The photonic examination approach often involves inserting optical fibers, typically disposed within an endoscope or similar device, into the patient's body in proximity to the area to be examined. The area to be examined is irradiated with light transmitted thereto by optical fibers, and the light from the irradiated area is collected and transmitted by optical fibers to a spectroscopic device or camera and computer for observation and analysis.

Over the past twenty years, many researchers have laid down a strong foundation to apply optical spectroscopy for disease diagnosis or blood information in laboratory bench scales. Examples of spectroscopy diagnoses are: Chance in U.S. Pat. No. 5,987,351 “Optical coupler for in vivo examination of biological tissue”, Alfano et al. in U.S. Pat. No. 6,615,068 “Technique for examining biological materials using diffuse reflectance spectroscopy and the kubelka-munk function”, Alfano et al. in U.S. Pat. No. 6,208,886 “Non-linear optical tomography of turbid media”, Alfano et al. in U.S. Pat. No. 6,091,985 “Detection of cancer and precancerous conditions in tissues and/or cells using native fluorescence excitation spectroscopy”, Georgakoudi et al in U.S. patent application No. 20030013973 “System and methods of fluorescence, reflectance and light scattering spectroscopy for measuring tissue characteristics”. Examples of methods in hemoglobin diagnosis are Schmitt, et al., in the “Measurement of Blood Hematocrit by Dual-wavelength Near-IR Photoplethysmography,” in SPIE proceedings in 1992 and Sodickson's “Kromoscopic Analysis: A Possible alternative to spectroscopic analysis for noninvasive measurement of analytes in vivo” in Clinical Chemistry magazine in 1994. These spectroscopic studies will be adapted with today's system integration technologies in our wireless spectroscopy biopsy capsule invention.

Optical spectroscopy from a tissue sample has been used in pathology to determine the disease in laboratory. With the advancement of today's photonic technology, broad-specttum light sources of laser diodes and LED (light emitting diode) are readily available and can be coupled into a mini-scale sensor or capsule. These broad spectrum and compact light sources can be configured and utilized with a variety of different fluorescence or absorption or diffuse reflect spectra. One or differing excitation wavelengths can be used in these approaches. The chemical and biological threats are detected and identified through interactions between the light and the matter.

A wireless capsule can be used to collect gastrointestinal (GI) tract tissue samples or other specimen of patients using special designed devices. The capsule comprises one or multiple LEDs, one or multiple optical information filter modules, one or multiple optical sensors, a signal-processing module, and a data storage module. The filter module is often coated on the surface of the optical sensor. The spectroscopy can also be used to measure the physiological and/or biochemical parameters in tissues and juices of a biological body, such as pH, osmolarity, temperature, ion concentrations, SaO₂, SaCO₂ hemoglobin, glucose, cholesterol, cholesterol esters, lipoproteins, triglyceride of changes in optical characteristic to diagnose the disease.

Alfano, R. et al. in U.S. Pat. No. 6,240,312 “Remote-controllable, micro-scale device for use in medical diagnosis and/or treatment”, revealed some basic concepts using spectroscopic diagnosis in a wireless capsule. They did not provide detail designs and methods such as biosensors or sample collection methods. Kim, et al in U.S. patent application No. 20030092964 “Micro capsule type robot” and Kimchy, et al in U.S. patent application No. 20030139661 “Ingestible device”, were aiming on the mechanical and optical designs of a wireless capsule.

One development of mini-scale sensors is the biosensor, which behaves as a miniature bio-probe and data processor. Biological data of the tissue sample can be analyzed either in vivo or in vitro (after the biosensor is discharged from the anus). A biosensor can be used to detect biomarkers, such as hepatocarcinoma-intestine-pancreas/pancreatitis-associated-protein I (HIP/PAP-I) in pancreatic juice for early diagnosis of pancreatic adenocarcinoma; the dyed antibody of p53 tumor suppressor gene in the GI wall for diagnosing the cancers; or any dye-marked target which has an optical characteristic. A biosensor can also be used to measure the physiological and/or biochemical parameters in GI juices, such as cholecystokinin-(26-33) (CCK-8), special proteins, and some changes in optical properties of GI tissues or GI juices for diagnosing disease and criticizing the GI physiological conditions.

### BRIEF SUMMARY OF THE INVENTION

It is an object of the present invention to provide a novel medical diagnosis tool that combines wireless capsule with micro-spectroscopy to detect morphology and/or chemical component changes inside a biological body in vivo.

It is an object of the present invention to provide a novel medical diagnosis tool that combines a wireless capsule with micro-biosensor to detect changes in DNAs, proteins, enzymes and antibodies inside a biological body in vivo.

It is an object of the present invention to provide a novel medical diagnosis tool that can collect one or multiple specimens inside a biological body guided by the information from micro-spectroscopy and/or micro-biosensor in vivo.

It is another object of the present invention to provide a novel medical diagnosis tool that combine one or multiple techniques described above to provide a multiple functional wireless capsule for medical uses.

As a result of extensive devolvement in order to achieve the above objects, the inventors further developed the above knowledge found by the inventors, and discovered that the above objects were accomplished by
1. “Optical coupler for in vivo examination of biological tissue”, Chance in U.S. Pat. No. 5,987,351.
2. “Technique for examining biological materials using diffuse reflectance spectroscopy and the kubelka-munk function”, Alfano et al. in U.S. Pat. No. 6, 615,068.
3. “Non-linear optical tomography of turbid media”, Alfano et al. in U.S. Pat. No. 6,208,886.
4. “Detection of cancer and precancerous conditions in tissues and/or cells using native fluorescence excitation spectroscopy”, Alfano et al. in U.S. Pat. No. 6,091,985.
5. “System and methods of fluorescence, reflectance and light scattering spectroscopy for measuring tissue characteristics”, Georgakoudi et al in U.S. patent application No. 20030013973.
6. “Measurement of Blood Hematocrit by Dual-wavelength Near-IR Photoplethysmography”, Schmitt, et al. in the in SPIE proceedings in 1992.
7. “Kromoscopic Analysis: A Possible alternative to spectroscopic analysis for noninvasive measurement of analytes in vivo”, Sodickson in Clinical. Chemistry magazine in 1994.

All of which are incorporated herein by reference.

This invention will integrate technologies of miniature light sources, light detector, biosensor, and remote sample collection, using disease sensitizing agents, optical spectroscopy and imaging to build a wireless capsule for non- or mini-invasive medical diagnoses.

The following detailed description is, therefore, not to be taken in a limiting sense, and the scope of the present invention is best defined by the appended claims.

### BRIEF DESCRIPTION OF THE DRAWINGS

The accompanying drawings, which are hereby incorporated into and constitute a part of this specification, illustrate preferred embodiments of the invention and, together with the description, serve to explain the principles of the invention. In the drawings wherein like reference numerals represent like parts:

FIG. 1 A schematic design diagram of a wireless imaging-spectroscopy capsule biopsy using a micro-spectrometer for the targets in tissues and/or juices.

FIG. 2 A schematic block diagram of a micro-spectrometer using N narrow-band filterlbeam splitter spectral signal detection. N is an integer number from 2 to 1000. Using several (1 to 10) LED illumination source, various optical signals can be generated from the specimen inside a collection chamber. The transmission or fluorescence optical signal will be collected through a filter module. The dispersed output will be measured by N photodiodes for N's distinct signal wavelengths. An example of a miniature grating is a spectrometer on a chip, which disperses different wavelengths into different positions of a detector array.

FIG. 3 A flow chart of a wireless capsule for in vivo biopsy.

FIG. 4 The first example of a biopsy capsule schematic design using LED (light emitting diode) for the absorption spectroscopy diagnosis of GI tract bleeding in vivo. Two LEDs that emit wavelengths at 660-nm and 940-nm, respectively, will be used as the illumination sources. A special designed hologram shown in FIG. 15 will be used to combine different illumination light sources and then collecting the signals at different wavelengths back scattered from tissues.

FIG. 5 The second example of a biopsy capsule schematic design using LED for the fluorescence and absorption spectroscopy diagnosis of GI tract cancer in vivo. Two LEDs that emit wavelengths at 290-nm and 325-nm, respectively, will be used as the illumination sources. A designed hologram [details shown in FIG. 14 will be used to combine different illumination light sources. The fluorescence signals emitted from the tissues at the wavelengths of 340-nm and 380-nm will be collected and analyzed to determine the tissue states of cancer in vivo.

FIG. 6 An example of a wireless biopsy capsule internal core of for spectroscopic imaging with two specimen collection functions.

FIG. 7 An example of the outside shell rack of capsule of a motorized rotation controllable inner core of a wireless biopsy capsule.

FIG. 8 A schematic design of motorized blades and storage assembly of specimen collection.

FIG. 9 The first example of the integrated optical module for light delivery and collection design using four sets of front-lens, mirror, and side-lens structure. Left part: side view of the module. Right part: A-A′ cross-section view of the module.

FIG. 10 3D drawing of the integrated optical module shown in FIG. 9 using four sets of front-lens, mirror, and side-lens structure. (A) and (B) are two different methods to mount the CCD chip in this module.

FIG. 11 The second example of the integrated optical module for optical delivery and collection design using four sets of front-lens, side-lens, mirror, rear-lens structure located in front space of capsule. Left part: side view of the module. Right part: A-A′ cross-section view of the module.

FIG. 12 3D drawing of the integrated optical module shown in FIG. 11 using four sets of front-lens, mirror, side-lens, and rear lens structure. (A) and (B) are two different methods to mount the CCD chip.

FIG. 13 Three examples of the spatial arrangement of light sources and detectors in the detector module of a spectroscopy biopsy capsule.

FIG. 14 A schematic design diagram of an optical multi-spectral remote image and biopsy device using a holographic optical element for wavelength splitting and recombination. A hologram [3] can combine several light sources at different wavelengths for the sample illumination and signal collections. Three light sources are shown in this diagram as: 1a (sold line), 1b (dashed line), and 1n (dotted line). The emitted signals from the object will be collected to optical detector [2]. The detector could be a single photo-diode, multiple photo-diodes, diode array, CCD, or CMOS detectors.

FIG. 15 An example of a biopsy capsule using holographic optical elements. CF: color filter. The center portion [19] of the disk hologram is used to focus the illumination light to the specimen. The rest part [21] and [23] of this disk hologram is used to collect the back-scattered and/or fluorescence signals of the specimen to the photo-detector.

### DETAILED DESCRIPTION

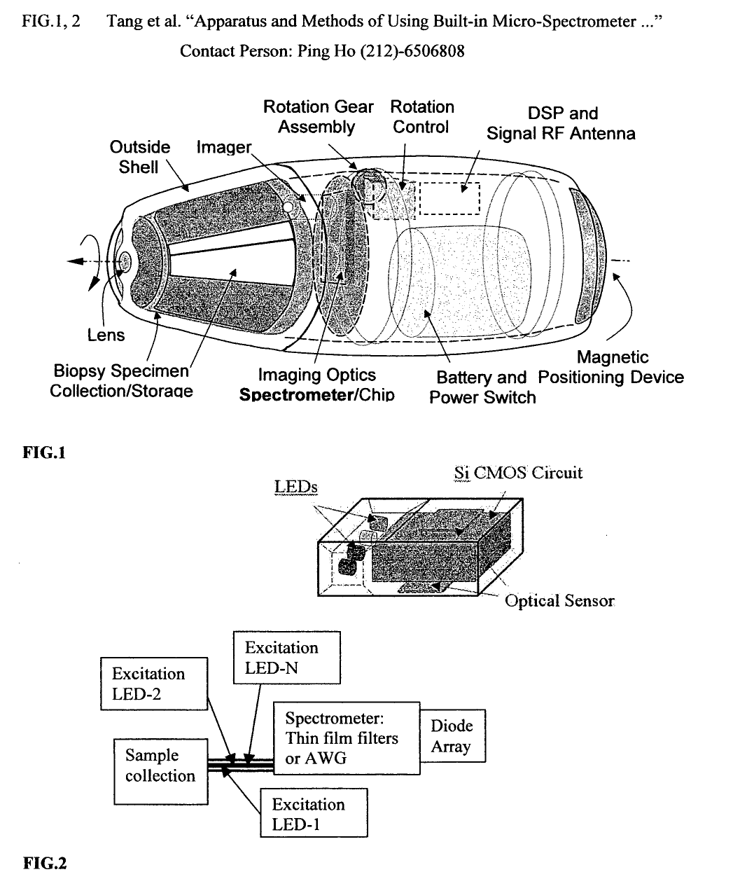

The principles and preferred embodiments of the present invention is a wireless capsule. A schematic diagram of a wireless capsule for spectroscopic biopsy is shown in FIG. 1. This capsule can travel into a nature tract of a living biological body, e.g., human body by a non-invasive or a minimally invasive procedure such as gastrointestinal (GI) by mouth, and urinary system, biliary tract, cardiovascular system by injection. Furthermore, this capsule can travel to a variety of sites inside the body, such as the esophagus, stomach, biliary tract, gallbladder, pancreatic tract, intestines, colon, rectum, urinary tract cardiovascular tract, and so on.

The wireless capsule adapted for use inside a biological body will be a capsule without a wire connection, but with or without a remote-control system outside the body. It will be 1 mm to 30 mm in length, 1 mm to 15 mm in wide or in diameter and a form as a cylinder or any other form. It comprises of
(a) a sheath of capsule;
(b) spectral or imaging means for collecting spectral or image information inside of the biological body;
(c) means for data analysis;
(d) means for indicating capsule position inside of the biological body;
(e) means for communication said transmitting information collected by spectral or imaging means or processed by data analysis means;
(f) with or without means of biosensors as a biological probe;
(g) with or without means of specimen collection.

The heart of the spectroscopy biopsy capsule is a micro-spectrometer. A design block diagram of a micro-spectrometer is shown in FIG. 2. The wireless capsule also includes a biosensor. The spectral dispersion component used in the micro-spectrometer can be either an array-waveguide-grating (AWG) for 2 to 128 wavelength channels, or a combined narrow-band filter/reflector for 2 to 4 wavelength channels, or one or multiple continuous wavelength ranges.

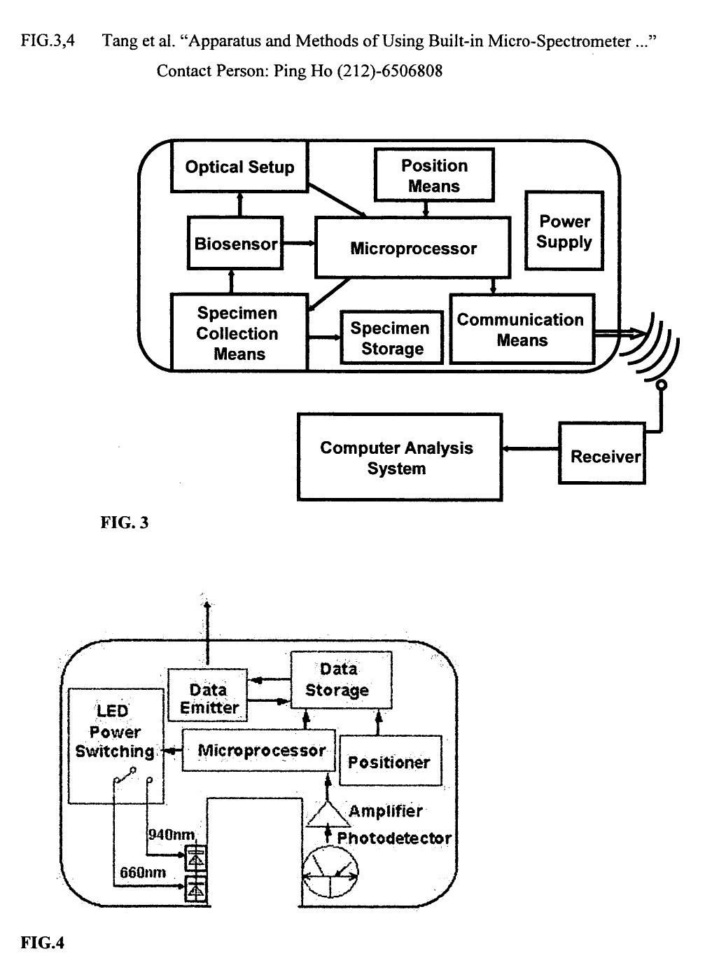

The wireless capsule consists of a specimen collection system, a spectroscopic system (comprising, for example, fluorescence-type and/or transmission-type and/or reflection-type gratings and filters), a motion mechanism, a communications system, a light source, an imaging system and a power system. A flow chart of the spectroscopic wireless capsule design is shown in FIG. 3. All of which are coupled to a microprocessor.

The foregoing devices can measure local tissue properties in situ using spectroscopic features from fluorescence, transmission, differ reflectance, scattering, and Raman bands. Two specific examples to detect GI bleeding and cancer are described below:

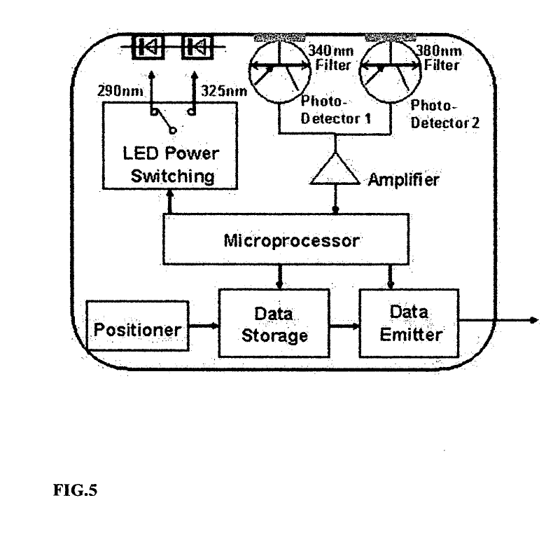

GI Bleeding Detection using Absorption Spectroscopy A wireless capsule comprises of a light emitting and light detecting parts as shown in FIG. 4. It can be swallowed through the mouth into GI system. The absorption spectra of GI juice will be observed continuously as the wireless capsule is traveling through the whole GI tract. The absorption spectra of GI juice can be obtained. A build-in position device will show the capsule position.

Both spectral and position data will be transmitted to a receiver belt worn on the human body. The final diagnosis will be performed by a computer system to compare the ratio of oxyhemoglobin and de-oxyhemoglobin concentrations. The maximum oxygenation value will reveal the bleeding area. Alternatively, a specimen storage module inside the capsule can save physically biopsy samples to be analyzed after the capsule is excreted from anus.

Cancer Diagnosis using Fluorescence Spectroscopy A wireless capsule can be designed for fluorescence spectroscopy. Major parts in this application include one or multiple light emitting diodes at different wavelengths and one or multiple photo detectors with selected wavelength narrow band filters as shown in FIG. 5. The size of the wireless capsule is small enough to be swallowed through the mouth into gastro-intestinal system (GI). The fluorescence intensities of one or more GI-cancer-sensitive wavelengths will be measured continuously, when the wireless capsule is traveling in the GI tract. The spectral data will be analyzed by a built-in microprocessor and then emitted to a receiver belt worn on of the patient body. A physician will perform the diagnosis using the computer processes data. The maximum or minimum ratio of different wavelength intensities of interest will indicate the cancer location. Similarly, a specimen storage module inside the capsule can save physically biopsy samples to be analyzed after the capsule is excreted from anus.

Other examples of using fluorescence spectroscopy to diagnose cancer are given by Alfano and co-workers using biopsy specimen in laboratory. Fluorescence spectra of normal tissues excited by 488 nm light were found to be quite different from that of cancer tissues. The emission spectra from cancer tissues have a smooth spectral curve with the peak at approximately 530 nm. The emission spectra from normal tissues have three peaks, at 530, 550, and 590 nm.

Operation procedures of using a wireless capsule to medical diagnosis is typically initiated through a native open such as through mouth by swallowing. It can also be launched from an endoscope, such as from a gastroscope into GI track and a cystoscope into bladder and urinary tract. After identified problems in GI tracts using a wireless imaging capsule, the second capsule is designed as a claim to collect diagnosis sample from the imaged location.

Procedures of capsule biopsy are described as follows:
a) The diagnosis capsule is performed in a doctor's office or in a hospital.
b) In order to reduce the battery power consumption, the battery will be on only when the capsule reaches the target location (measured by the first imaging capsule)
c) The capsule will be real-time monitored for positioning
d) The capsule will be accurately positioned and directional controlled
e) The special cutter in the capsule will collect specimen and tightly seal in sample storage space as shown in FIG. 7. Examples include a cup type device with sharp diamond blade, chain saw, at the rim. Collecting samples can also use a hollow drill.
f) Require methods for capsule specimen collection: send RF signals before the toilet (a receiver belt worn by the patient to detect the magnet as it arrives rectum or anus).
g) After a suspicious area, such as a polyp, bleeding, or color changes, has been identified, a diagnosis procedure will be performed either by the same capsule or a second diagnosis capsule to be delivered.

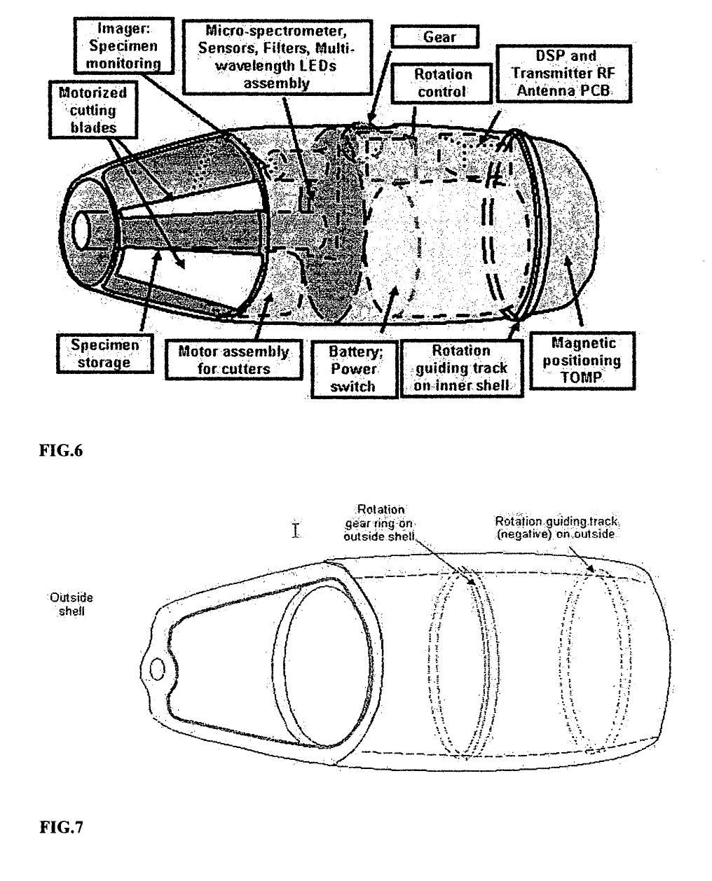

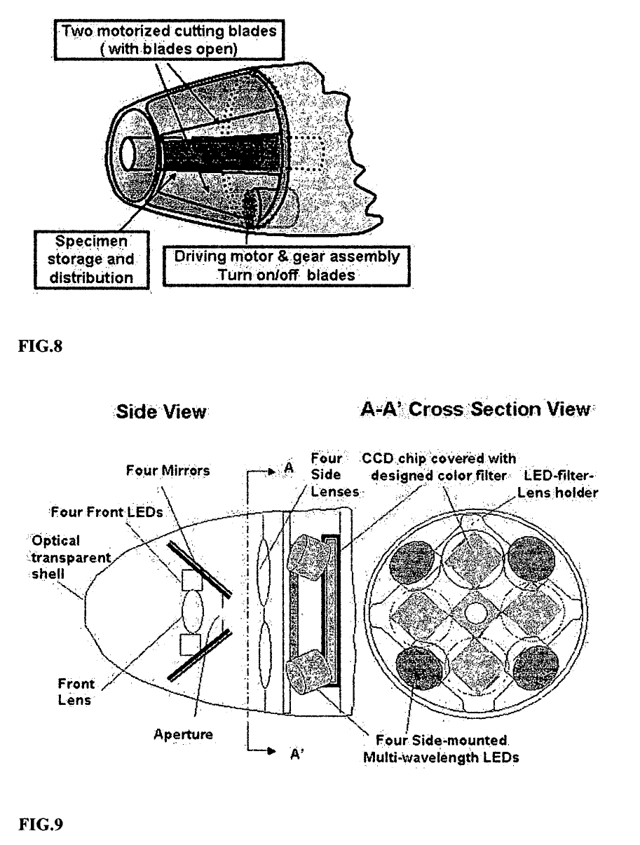

The solid tissue collection assembly has capabilities to adjust capsule position and azimuth status. Some components designed for the solid tissue specimen collection in a wireless capsule are shown in FIGS. 6 to 8 These parts include:
1. A rotation monitoring and control using a motor to drive inner core to move the inside and the outside shell racks;
2. A directional monitoring and control using the built-in magnet and an external magnetic field;
3. Sampling and monitoring the opening and closing of two motorized driving blades;
4. Two specimens can be collected simultaneously with one on each side of the capsule;
5. Specimen storage separately for each collection;
6. When two cutting blades are closed. It becomes a sealed storage space;
7. A pin-hole imager to monitor the specimen collection.

Liquid specimen collection can be performed using various methods, such as needles, reverse osmosis, permeation, porous structure, fiber structure, and hollow fibers.

Optical system for illumination and signal collection uses a multi-lens-mirror imaging assembly for the spectroscopy wireless capsule. The assembly consists of lenses, mirrors, LEDs, apertures, filters, and holders. The detection assembly uses either CCD or CMOS imaging chip.

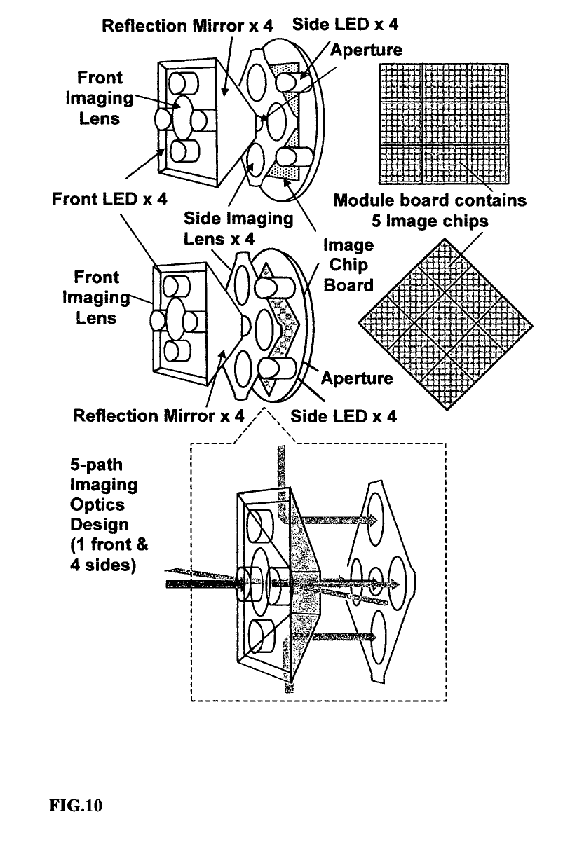

The first type is a combination of four sets of front-lens, side-lens, mirror, and rear-lens structure. The side view and the A-A′ cross-section view are shown in FIG. 9. The corresponding 3D drawings with the CCD chip amounted in two different methods are shown in FIGS. 10A and 10B, respectively.

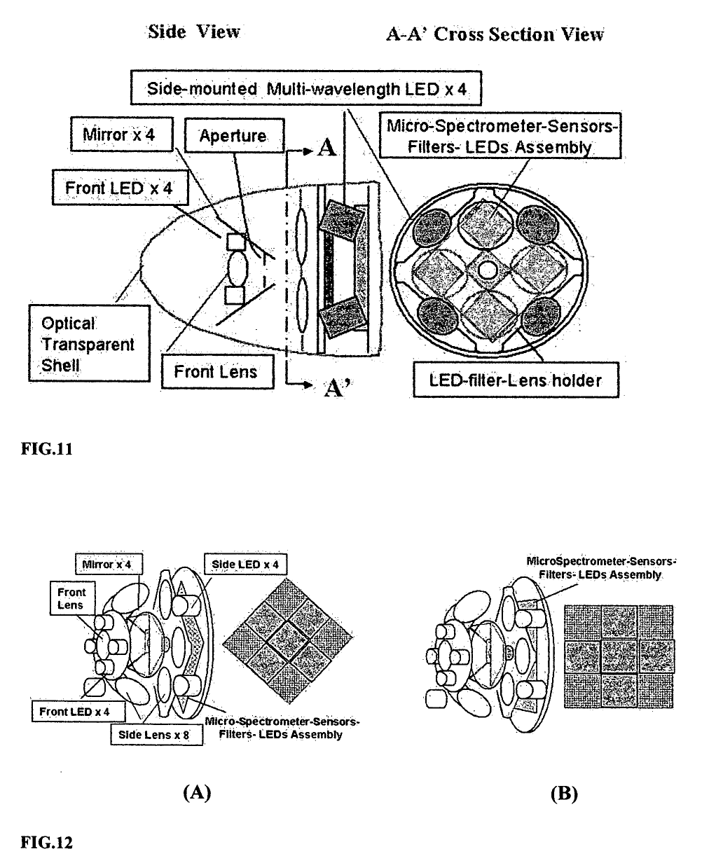

The second type is a combination of four sets of front-lens, mirror, and side-lens structure. The side view and the A-A′ cross-section view are shown in FIG. 11. The corresponding 3D drawings of FIG. 11 with the CCD chip amounted in two different methods are shown in FIGS. 12A and 12B, respectively.

Mirrors are optical reflection surfaces with positive, negative or zero curvature, i.e., concave, convex, or plane reflection surface. LED spectrum covers from the infrared to UV band. The combination of a front lens and the front surface of the optical shell can increase the field of view of imaging. A CCD chip or a CMOS chip is shared by five independent sets of imaging optics, including one wide-angle front imaging and four side high resolution imaging mechanisms.

The light source is preferably one or more micro-scale, color LEDs, lasers based on quantum wells or a photographic flash lamp. The combination two to three LEDs using a hologram can form a wideband light source with a controlled spectral intensity distribution. A combined uv LEDs (wavelength from 250-nm to 350-nm) with white light source will be used in bio-sensor applications inside a wireless capsule.

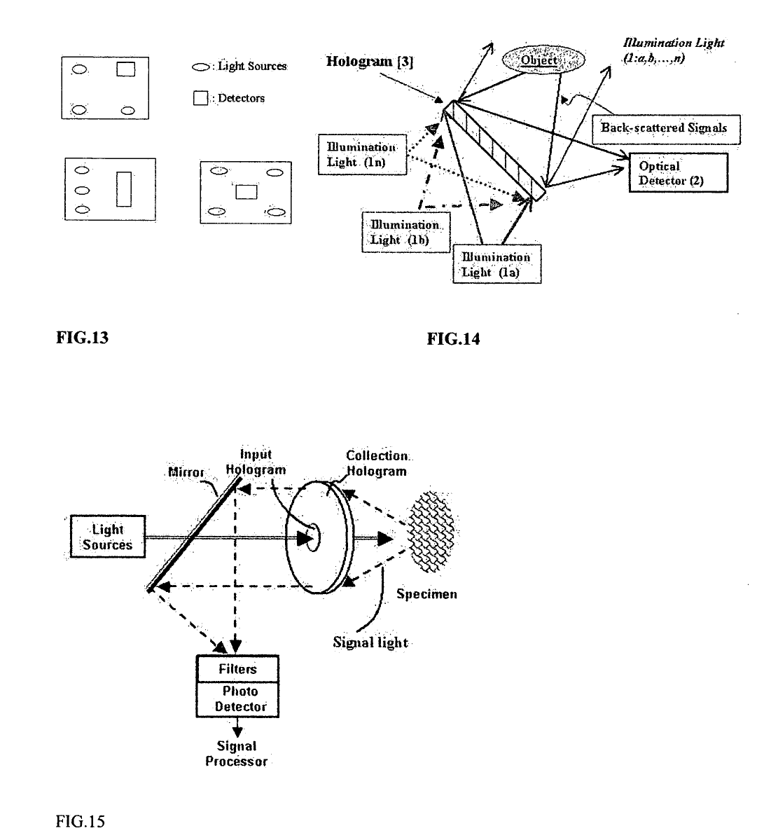

Optical detectors used in this invention for spectroscopy can be: a CCD or a CMOS chip with the pixel number from 10×10 to 4000×4000 and the spectral spanned from 300-nm to 1100-nm; a NIR camera with the pixels number from 10×10to 2000×2000 and the spectral sensitivity from 400-nm to 1800-nm. For one-dimensional detectors: PIN diode with spectral range from 300-nm to 1800-nm; or APD with spectral range from 300-nm to 1800-nm. Three examples of the position of light source and detector are shown in FIG. 13.

A hologram can perform several functions together: multi-function lenses, color filters, spectral reformer, and beam splitter. For the lens application using a hologram, light can be collimated to illuminate the specimen. An example of the holographic multi-function design is shown in FIG. 14. Collection of the back-scattered light from the object by a hologram can be tightly imaged to an optical detector. An example of the holographic optical delivery and collection design is shown in FIG. 15.

A spectral reformer can adjust the intensity spectral distribution of the illumination to match white light spectrum, Mercury arc spectrum, or sun light spectrum. A holographic beam splitter can provide high throughput efficiency for the illumination light transmission and the signal light reflection based on the geometrical factor and wavelength. By rotating a hologram, tunable narrow band filtering is obtained. This change of the effective grating space will be used as a re-configurable narrow band color filter for signal collection.

For the spectral reformer, the spectral intensity distribution could be determined using the following equation:
I[output, λ]=A₁I₁(λ₁±Δλ₁)+A₂I₂(λ₂±Δλ₂)+ . . . +AᵢIᵢ(λᵢ±Δλᵢ)+ . . . +AₙIₙ(λₙ±Δλₙ)
Where A₁,A₂ , . . . Aᵢ, . . . Aₙ are constant parameters and could be numerically optimized to fit the desired spectral intensity distribution; Iᵢ is the intensity of the i-th light source at the peak wavelength of λ_{I} with the bandwidth of Δλ_{I}, respectively.

After introduced into the inside of a biological body, the wireless capsule will function as a diagnosis modality. Besides two examples shown in FIGS. 4 and 5, other examples using different spectroscopic methods for clinical diagnoses are listed in Table. 1 below:

TABLE 1
Methods and Wavelengths for Spectroscopy Disease Diagnosis

| Disease | Method | Wavelength |
|---|---|---|
| GI pre- | Absorption | 400 to 440, 540 to |
| cancerous | | 580 nm scan |
| lesion | | |
| Esophageal | Fluorescence by an OMA | 410 nm excitation |
| cancer | | |
| Upper GI | Fluorescence, I₃₃₀/I_{380 nm} | 290, 330 nm |
| cancer | ratio | excitation |
| | Fluorescence by an OMA | 410 nm excitation |
| Colon | Fluorescence, I₆₀₀/I_{680 nm} | 370 nm excitation |
| cancer | ratio | |
| Cervical | Raman, I₁₆₅₆/I_{1454 cm-1} | 780 nm excitation |
| precancerous | I₁₄₅₄/I_{1330 cm-1} ratios, | |
| tissue | | |
| Cervical | Fluorescence | 337 nm excitation |
| cancer | FT-Raman, I₁₆₅₇ < I_{1445 cm-1} | 780 nm excitation |
| Bladder | Fluorescence by an OMA | 308, 337, 480 nm |
| cancer | | excitation |
| | Elastic-scattering | 330 to 370 nm |
| | | scan |
| Breast | FT-Raman, 1445, 1651 cm⁻¹ peaks | 780 nm excitation |
| cancer | Raman, I₁₄₃₉/I_{1654 cm-1} ratio | 784 nm excitation |
| Athero- | Fluorescence, reduce of I₄₆₀ | 248 nm excitation |
| sclerosis | Fluorescence, 340, 380 nm | 306 to 310 nm |
| | peaks | excitation |
| | Fluorescence, I₄₂₀/I_{480 nm} | 325 nm excitation |
| | peaks | |

The other examples of detecting optical properties changes of solid tissue or juice in GI tract is that optical absorption spectra can be recorded simultaneously and continuously in the pancreas arterially perfused at various flow rates. This is done to explain how optical absorbance changes corresponding to parallel reduction of cytochromes aa3, b, and cc1 are observed in perfused pancreas stimulated with high concentration of an exocrine secretagogue, such as cholecystokinin-(26-33) (CCK-8). With perfusion flow rate between 1.5 and 3.0 ml/min, there are no optical absorbance changes corresponding to cytochrome reduction, but these optical absorbance changes occur when the perfusion flow rate is decreased to 1.0 ml/min. These optical absorbance changes are not observed during exocrine secretion stimulated by CCK-8 at the perfusion flow rate of 3.0 ml/min.

Transient but a slight change in optical absorbance, which corresponds to reduction of cytochromes, is observed in the glands perfused at the flow rate of 2.0 ml/min when secretion is stimulated by 1 nM CCK-8. When the perfusion flow rate is decreased to 1.0-1.5 ml/min, these optical absorbance changes corresponding to reduction of cytochromes occurred in glands stimulated by CCK-8. Optical absorbance changes corresponding to reduction of mitochondrial cytochromes during secretion stimulated with CCK-8 may indicate local hypoxia in the perfused organ.

Other examples of tissue and/or juice optical property measurements for clinical diagnosis are listed in Table 2 below:

TABLE 2
Measurement of Tissue and/or Juice Optical
Properties for Disease Diagnosis

| Target | |
|---|---|
| Detected | Method |
| Hemoglobin | Transmission, diffuse reflect, life time fluorescence |
| | spectroscopy |
| PH | Diffuse reflect, life time fluorescence spectroscopy |
| Oxygenation | Transmission, diffuse reflect, life time fluorescence |
| | spectroscopy |
| Bilirubin | Reflectance spectroscopy |
| Drug | Single photon emission computer tomography, positron |
| concentration | emission tomography |

Light-induced fluorescence of exogenous fluorophores can be performed using a wireless biopsy capsule. An example of this application is injecting photofrin as a photosensitized dye into living body 48 h before spectroscopy. The wireless capsule will be inserted into the bladder via a cystoscope. Fluorescence was taken and a ratio of red photosensitized dye fluorescence to the blue auto-fluorescence of the tissue will be calculated. Based upon this ratio, excellent demarcation between papillary tumors and normal bladder wall will be achieved.

Swallow sensitized dyes for in vivo capsule biopsy in GI tract disease and/or functions. The examination using a wireless capsule can be performed in a physician's office or in a hospital. The methods of providing dye include swallow, IM injection, IV injection, and local inunctions. The wireless capsule can be introduced into the native track of the biological body through swallow, injection or an endoscope. For example: the wireless capsule can be swallowed into the GI track via mouth; can be shot into the cardiovascular system via percutanous injection; and can be inserted into the bladder via a cystoscope.

Examples of different spectroscopy with exogenous dyes for clinical diagnoses are listed in Table 3:

TABLE 3
Examples of Photosensitized Dyes for Disease Diagnoses

| Dyes | Diseases Diagnosed/Treated | Wavelength |
|---|---|---|
| Indocyanine green (ICG) | Brain tumor | 790, 805 nm |
| Pure hematoporphyrin Hp/5 | Gastrointestinal tumors | 630 nm |
| HEMATODREX (Bulgarian | Gastrointestinal tumors | 630 nm |
| hematoporphyrin derivative) | | |
| Haematoporphyrin derivative | Advanced gastrointestinal | Argon dye |
| (HpD) | cancers | laser |
| Haematoporphyrin | Central bronchial carci- | 628.2-630 |
| | noma and gastrointestinal | nm |
| | tract (oesophageal and | |
| | colonic) early-stage | |
| | cancer | |
| Pure hematoporphyrin | Cancers of esophagus, | 630 nm |
| | stomach, rectum | |
| Photofrin | Esophageal, intraperi- | 532 nm, 630 |
| | toneal tumors, gastro | nm |
| | intestinal, lung, skin, | |
| | brain early adeno- | |
| | carcinoma | |
| Phototoxic drug (HPD) | Gastrointestinal | 632 nm |
| | tumors | |
| Porfimer sodium | Esophageal varies | Argon-dye |
| | | laser (630 |
| | | nm) |
| Meso-tetrahydroxy phenyl | Pancreatic cancer | Blue |
| chlorin | | |
| 5-aminolevulinic acid | Small gastrointestinal | 380-450 |
| (ALA) | tumor | nm |
| 5-aminolevulinic acid-induced | dysplastic Barrett's | Blue (peak |
| protoporphyrin IX, ALA | oesophagus | at 417 nm) |
| thermosetting gel Pluronic | | |
| F-127 | | |
| 5-aminolevulinic acid esters | Adenocarcinoma | Blue |
| on protoporphyrin IX | | |
| 5-aminolevulinic acid-in- | Low- or high-grade | Blue |
| duced protoporphyrin IX | dysplasia Barrett's | |
| | esophagus | |
| Meso-tetrahydroxyphenyl- | Oral, gastrointestinal | 650 nm |
| chlorin | tract | |
| pyropheophorbide-alpha- | Lung, esophagus, | 665 nm |
| hexyl-ether (HPPH-23). | gastrointestinal | |
| | cancer | |

Biosensor technology is also coupled into this biopsy capsule invention. The publication of Jin, et al. “Voltage sensitive dye imaging of population neuronal activity in cortical tissue,” in J. Neuroscience Methods in 2002 provides a good example of the voltage enhanced dye imaging approach. Sadoulet's article in the magazine of Biophotonics International “Using light to read the code of life”, in 2003 gave a good review of those miniature spectrometer technology. McMullin, et al. in “Optical Detection System for biosensors using Plastic Fiber Optics” (2003), Thrush et al. in “Integrated semiconductor fluorescent detection system for biosensor and biomedical applications,” (2003) and Ting, et al. in “Research and development of biosensor technologies in Taiwan,” (2000) have provided the design and integration of biosensors with various optical technologies.

Fast and sensitive detection of K-ras mutations in tumor cells of GI tracts are attractive targets for molecular screening and early detection of colon or pancreatic malignancies. Using a biosensor and an optical transducer could be performed.

An example of chemi-luminescence (CE) detection in a flow-thru wireless capsule in vivo can increase both the sensitivity and spatial. Enzyme-catalyzed CL reactions for the detection of hybridizations can be imaged using a CCD camera. Similar to two-color fluorescence measurements, multiple enzyme labels can be used. Relaxation time of a CL species can be applied.

Alterations in the gene have been associated with carcinogenic manifestations in several tissue types in humans. The design of this highly integrated detector system is based on miniaturized phototransistors having multiple optical sensing elements, amplifiers, discriminators, and logic circuitry in a wireless capsule. The system utilizes laser or LED excitation and fluorescence signals to detect complex formation between the p53 monoclonal antibody and the p53 antigen. Recognition antibodies are immobilized on a nylon membrane platform and incubated in solutions containing antigens labeled with Cy5, a fluorescent cyanine dye. Subsequently, this membrane is placed on the detection platform of the biosensor and fluorescence signal is induced using a 632.8-nm He—Ne laser or LED. Using this immuno-biosensor, we have been able to detect binding of the p53 monoclonal antibody to the human p53 cancer protein in biological matrices. The performance of the integrated phototransistors and amplifier circuits of the biosensor, previously evaluated through measurement of the signal output response for various concentrations of fluorescein-labeled molecules, have illustrated the linearity of the microchip necessary for quantitative analysis. The design of this wireless capsule permits sensitive, selective and direct measurements of a variety of antigen-antibody formations at very low concentrations.

Other examples of biosensor diagnoses are listed in Table 4 below:

TABLE 4
Examples of Biosensor Diagnosis

| Type of | | |
|---|---|---|
| Biosensor | Measurement | Disease/Objective |
| Nucleic acids/ | HIV1 gene fragments | AIDS |
| DNA | BRCA 1 BRCA2, p35, p450 | Cancers, |
| Antibody/anti- | Protein A | Staphylococcus aureus |
| gen | | infection |
| | Prostate-specific antigen | Prostate cancer |
| | (PSA) | |
| | Carcinogen benzo [a] | Cancers |
| | pyrenc (BaP) | |
| | E. coli via Cy5-labeled | E. coli infection |
| | antibody | |
| Enzymes | Base on pH changes | Detection of Penicillin |
| | | and Ampicillin |
| | Base on enzyme reaction | Detection of glucose |
| Cellular | Staphylococcus aureus stain | Staphylococcus aureus |
| structure/cells | (Wood-46) | infection |
| | Herpes simplex virus type 1 | Herpes infection |
| | (HSV-1), type 2 (HSV-2) | |

Specimen collection system in the wireless capsule is described as: The biomarkers of pancreatic adenocarcinoma improve the early detection of this deadly disease to screen for differentially expressed proteins in pancreatic juice (Cancer Res 2002 Mar. 15; 62(6):1868-75, Rosty C, et al.). Pancreatic juice samples can be obtained from patients via a sallow-able wireless capsule. The differentially expressed protein as hepatocarcinoma-intestine-pancreas/pancreatitis-associated-protein I (HIP/PAP-I), a protein released from pancreatic tract during acute pancreatitis and over expressed in hepatocellular carcinoma.

Another application using the specimen collection system is for the pharmaceutical and pharmacological study. The wireless capsule can collect specific specimen from a specific region, e.g. gastric juice in the stomach and pancreatic juice in the duodenum. The collection procedure can be programmed by a microprocessor inside the wireless capsule. The collection can also be performed by a feedback from the spectroscopy or biosensor inside the wireless capsule or by an external trigger signal from outside the human body.

1. A wireless capsule that used inside a biological body as a diagnosis tool in vivo comprises
a) Examining means for medical diagnosis;
b) Means for specimen collection;
c) Means for positions and trace;
d) A microprocessor for data storage, data analysis, data transmission and system control;
e) Means for communication to outside of the biological body;
f) A protect capsule.

2. The wireless capsule claimed in claim 1 wherein said the biological body in vivo is a living human or a living animal.

3. The wireless capsule claimed in claim 1 wherein the inside of a biological body is:
a. Gastrointestinal tract,
b. Biliary tract;
c. Pancreatic tract;
d. Breast ducts;
e. Urinary tract;
f. GYN tract;
g. Brain ventricular system;
h. Cardiovascular system.

4. The wireless capsule claimed in claim 1 wherein said examining means is a micro-spectrometer and/or a micro-biosensor with a microprocessor.

5. The wireless capsule claimed in claim 1 wherein said the micro-spectrometer claimed in claim 4 comprises a light source for illumining an area inside biological body or a micro-biosensor, an optical sensor for detecting light from the irradiated area and other optical assistances at one or multiple wavelengths. The micro-spectrometer comprises a set of beam splitter/narrow band filter set as shown in FIG.2 or an array-wavelength-grating to disperse different wavelengths into different detectors.

6. The wireless capsule claimed in claim 5 wherein said light source is a broad-spectrum light of light emitting diode (LED), laser diode, or flash lamp or tunable diode lasers with or without wavelength selection filters covering wavelength range from 190 nm to 2500 nm.

7. The wireless capsule claimed in claim 6 wherein said LED is
a. LED (spectral bandwidth <100 nm): the peak illumination wavelength spans from 280 nm to 2500 nm;
b. LED (spectral bandwidth >300 nm): the peak illumination wavelength spans from 280 nm to 2500 nm;
c. LED as a white light source (5900 K black body radiation) as the Mercury arc lamp;
d. Laser diode whose peak emission wavelength spans from 250 nm to 2500 nm;
e. Combination of several LEDs or laser diodes using a hologram to form a wideband light source with a controlled spectral intensity distribution;
f. Combined NIR LEDs or laser diodes with white light sources using holograms to perform white light source;
g. Combined UV LEDs (wavelength from 190 nm to 350 nm) with white light source.

8. The wireless capsule claimed in claim 5 wherein said the optical sensor is configured with a variety of image and/or different fluorescence and/or absorption and/or diffuse reflect and/or transmission spectra, which have one or differing excitation wavelengths to detect chemical and biological threats, or as an optical transducer for biosensors, or as an indicator for specimen collection in vivo.

9. The wireless capsule claimed in claim 5 wherein said the optical sensor comprises one of:
a. One or multiple photodiodes;
b. One or multiple photomultipliers;
c. A CCD chip with pixel size: 10×10 to 4000×4000, spectral spanned from 190 nm to 2500 nm;
d. A CCD chip shared by five independent sets of imaging optics, including one wide-angle front imaging and four side high resolution imaging mechanics;
e. A CMOS imaging chip: pixel size: 10×10 to 4000×4000, spectral spanned from 190 nm to 1100 nm;
f. A NIR camera: pixel size: 10×10 to 2000×2000, spectral sensitivity from 800 nm to 2500 nm;
g. One or multiple PIN diodes with spectral range from 190 nm to 2500 nm;
h. One or multiple avalanched photodiodes (APD) with spectral range from 190 nm to 2500 nm;
i. A diode array with the total number of diodes from 10 to 8000 and the spectral range from 190 nm to 2500 mn.

10. The wireless capsule claimed in claim 5 wherein said the other optical assistance is:
a. Lenses: Collimation of the illumination light source to illuminate the object, collection of the back-scattered light from the object and image to the optical detector, collection of the transmission light from the object and image to the optical detector, collection of the fluorescence light from the object and image to the optical detector;
b. Color filters: Narrowband filters with the center wavelength spanned from 190 nm to 2500 nm, broadband filters with the center wavelength spanned from 190 nm to 2500 nm;
c. Polarization filters covering the wavelength range from 190 nm to 2500 nm;
d. Spectral reformer: adjust the intensity spectral distribution of the illumination to match white light spectrum, mercury arc spectrum, sun light spectrum;
e. Beam splitters: high throughput efficiency for the illumination light transmission and the signal light reflection based on the geometrical factor and wavelength;
f. Tunable narrow band filters: by rotating the hologram, the change of the effective grating space as a re-configurable narrow band color filter for signal collection.

11. The wireless capsule claimed in claim 10 wherein said the lens is:
a. A single lens;
b. A combination of four sets of Front-lens Side-lens Mirror Rear-lens structure (The side view and cross-section view is shown in FIG. 9, and 3D drawing is shown in FIGS. 10A and 10B), where the CCD chip amounted in two different ways;
c. A combination of four sets of Front-lens Mirror Side-lens structure. (The side view and cross-section view is shown in FIG. 11, and 3D drawing is shown in FIGS. 12A and 12B with different ways to mount CCD chip);
d. A combination of front lens and the spatial surface profile of front part of optical shell widens range of imaging angle.

12. The wireless capsule claimed in claim 1 wherein said the optical transducer for biosensor is a micro-spectrometer, described in claim 5, with a microprocessor.

13. The wireless capsule claimed in claim 4 wherein said the biosensor is one of:
a. A DNA chip;
b. An enzyme chip;
c. An antibody chip;
d. A cell or cellular system chip;
e. A bio-mimetic chip;
f. A set of micro-sphere sensors;
g. A micro-array smart pin sensor.

14. The wireless capsule claimed in claim 1 wherein said the biosensor has one or multiple sets in the wireless capsule for different area detection.

15. The wireless capsule claimed in claim 1 wherein said the biosensor:
a. Without a transducer;
b. With an optical transducer;
c. With an electrochemical transducer; or
d. With a Mass-based transducer.

16. The wireless capsule as claimed in claim 1 wherein said the means for specimen collection is controlled by examining means, described in claim 4, with a microprocessor.

17. The wireless capsule as claimed in claim 1 wherein said the specimen is liquid or solid samples.
Specimen collection means for solid samples are:
a. A cup-type device with sharp diamond blade or chain saw at the rim;
b. A hollow drill.
Specimen collection means for liquid samples are:
a. Needles;
b. Hollow fibers;
c. Reverse osmosis;
d. Permeation;
e. Porous structure.

18. The wireless capsule as claimed in claim 1 wherein said the protect capsule has a length from 1 mm up to 30 mm and has any form. The protect capsule is made of plastic, Teflon, silicon and/or metal.

19. The wireless capsule as claimed in claim 1 wherein said the communication means is a system of emitting electromagnetic waves, using radio frequency (RF). The position and trace information of a said capsule to a receiver outside of the biological body is using electromagnetic fields and waves from:
a. RF;
b. Magnet;
c. Radio-Isotope.

20. The wireless capsule as claimed in claim 1 wherein said the microprocessor is:
a. Micro-spectrometer system;
b. VLSI circuit;
c. Si CMOS circuit;
d. Any semiconductor chip.

21. A system for diagnosis in internally a biological body with wireless capsule, said the system comprises:
a. Means for receiving wireless signals;
b. A computer with software for analyzing wireless signals; and
c. Wireless capsule as claimed in claim 1.

22. A method of diagnoses diseases comprises the steps of:
a. Providing the wireless capsule claimed in claim 1;
b. Providing a photosensitized dye and/or other drug or not;
c. Introducing the wireless capsule into the biological body;
d. Collecting examination information through the microprocessor via micro-spectroscopy and/or micro-biosensor;
e. Transmitting the examination information to a receiver located outside a biological body; or
f. Collecting specimen indicated by the exanimation information obtained.

23. The method as claimed in claim 22 wherein said introducing the wireless capsule into the biological body is via:
a. A native open;
b. An artificial open;
c. An endoscope;
d. An injection.

24. The method as claimed in claim 22 wherein diagnosing diseases through the wireless capsule inside of a biological body in vivo is:
a. Spectroscopy;
b. Imaging;
c. Biosensor with or without a transducer;
d. Collecting the specimen for further outside analysis.

25. The method as claimed in claim 22 wherein said examining information is:
a. Day light imaging of tissue and/or juice;
b. Scatter spectra and/or imaging of tissue and/or juice;
c. Absorption spectra and/or imaging of tissue and/or juice;
d. Transmission spectra and/or imaging of tissue and/or juice;
e. Fluorescence spectra and/or imaging of tissue and/or juice;
f. Raman spectra and/or imaging of tissue and/or juice;
g. Differ and reflectance spectra and/or imaging of tissue and/or juice;
h. Time-resolved spectra and/or imaging of tissue and/or juice;
i. DNA analyses of tissue and/or juice;
j. RNA analyses of tissue and/or juice;
k. Protein analyses of tissue and/or juice;
l. Antibody analyses of tissue and/or juice;
m. Enzyme analyses of tissue and/or juice;
n. Cell and/or cellular system analyses of tissue and/or juice;
o. pH analysis of tissue and/or juice;
p. Osmolarity analysis of tissue and/or juice;
q. Temperature analysis of tissue and/or juice;
r. Ion concentration analyses of tissue and/or juice;
s. SaO₂ analysis of tissue and/or juice;
t. SaCO₂ o analysis f tissue and/or juice;
u. Hemoglobin analysis of tissue and/or juice;
v. Glucose analysis of tissue and/or juice;
w. Cholesterol analysis of tissue and/or juice;
x. Cholesterol esters analysis of tissue and/or juice;
y. Lipoproteins analysis of tissue and/or juice;
z. Triglyceride analysis of tissue and/or juice;
aa. Any other physiological parameter analysis of tissue and/or juice.

26. The method as claimed in claim 22 wherein said a photosensitized dye is:
a. ICG;
b. Pure hematoporphyrin Hp/5;
c. HEMATODREX (Bulgarian hematoporphyrin derivative);
d. Photofrin;
e. Pure hematoporphyrin;
f. Hematoporphyrin derivative (HpD);
g. Hematoporphyrin;
h. Phototoxic drug;
i. Porfimer sodium;
j. Meso-tetrahydroxyphenyl chlorine;
k. 5-aminolevulinic acid (ALA)-induced protoporphyrin IX, ALA thermosetting gel Pluronic F-127;
l. 5-aminolevulinic acid esters on protoporphyrin IX;
m. 5-aminolevulinic acid;
n. 5-aminolevulinic acid-induced protoporphyrin IX;
o. Meso-tetrahydroxyphenylchlorin;
p. Pyropheophorbide-alpha-hexyl-ether (HPPH-23);
q. Di-sulphonated aluminium phthalocyanine (A1S2Pc).

27. The method as claimed in claim 26 wherein said providing a photosensitized dyes is:
a. Swallow;
b. Injection;
c. Local provided.

28. The method as claimed in claim 24 wherein said using spectroscopy is using spectral analysis of:
a. Scattering: Given an illumination source of Iᵢₙ(λ₁) with known intensity and wavelength, the output Iₒᵤₜ(λ₁) has the same wavelength using the function of amplitude, angular distribute, and/or polarization information to determine diseases;
b. Absorption: Using N illumination sources of Iᵢₙ(λ₁), Iᵢₙ(λ₂), . . . Iᵢₙ(λ_{N}) with known intensity, the measured intensity change from the output Iₒᵤₜ(λ₁), Iₒᵤₜ(λ₂), . . . Iₒᵤₜ(λ_{N}) will be collected and normalized with Iᵢₙ to determine diseases. N is an integer number greater or equal 2;
c. Fluorescence: Given an illumination source of Iᵢₙ(λ₁) with known intensity and wavelength, Iᵢₙ(λ₁) the output intensities at different wavelengths, Iₒᵤₜ(λ_{F}_{₁},), . . . Iₒᵤₜ(λ_{F}_{_{N}}) will be measured and analyzed to determine diseases;
d. Excitation: Using N illumination sources of Iᵢₙ(λ₁), Iᵢₙ(λ₂), . . . Iᵢₙ(λ_{N}) with known intensity, Iᵢₙ, and wavelength, λᵢ, then measure the output intensities emission at a particular wavelength (Iₒᵤₜ(λₚ),) illuminated from various input wavelength (λᵢ) to determine diseases. i is an integer number from 1 to N;
e. Raman: Using one illumination source of Iᵢₙ(λ₁) with known intensity and wavelength, the output signals at various phonon vibration wavelengths (λ_{R}_{ᵢ}) will be measured to determine the chemical compositions of each molecular chain. λ_{R}_{ᵢ} is the i-th Raman signal wavelength and i is an integer number from 1 to N. The larger the N is, the more accurate disease information will be obtained;
f. Nonlinear: Using one illumination source of Iᵢₙ(λ₁) with known intensity and wavelength, the output signals at various high order harmonic generation wavelengths (λ_{R/i}) will be measured to reveal tissue structural behaviors. i is an integer number from 1 to N. For example, the wavelength of the second harmonic generation is λ/2, and the third harmonic generation is λ/3, and the n-th harmonic generation is λ/N;
g. Time-resolved: Using a pulsed illumination source of Iᵢₙ(λ₁, t), the output signal intensity at a particular wavelength, λ_{F}, will be measured as a function of time: t₁, . . . , t_{N};
h. Beam Forming Optics: using both diffuser and hologram: holographic Optical Elements as multi-function lenses, color filters, spectral reformer, beam splitter for illumination light focusing, signal light collection, and wavelength spectral correction;
i. Apply provided photosensitized dyes for different spectral analyses.

29. The method as claimed in claim 24 wherein said imaging is:
a. Day light imaging;
b. Fluorescence imaging;
c. Absorption imaging;
d. Scatter imaging;
e. Time-resolved imaging;
f. Hologram;
g. Thermal imaging;
h. Pseudo color imaging.

30. The method as claimed in claim 22 wherein said using biosensor with or without an optical transducer is:
a. Hepatocarcinoma-intestine-pancreas/pancreatitis-associated-protein I (HIP/PAP-I) in pancreatic juice for early diagnosis of pancreatic adenocarcinoma;
b. Human express sequence tags (ESTs) for lung and prostate cancers;
c. Single-nucleotide polymorphism (SNP) for cancer, diabetes, vascular disease and some forms of mental illness;
d. Loss of heterozygosity (LOH) for human tumors;
e. Human genes BRCA1 and BRCA 2, p53, p450 for cancers;
f. Comparative genomic hybridization (CGH) data for ovarian, prostate, breast, urinary bladder caner and renal cell carcinoma;
g. The dyed antibody of p53 tumor suppressor gene in the GI wall for cancer diagnoses;
h. Any dye-marked target that has an optical characteristic.

31. The method as claimed in claim 28 wherein said the method of collecting specimen can be controlled by:
a. An indication from examination means inside of wireless capsule;
b. An program from microprocessor inside of wireless capsule;
c. An order from the outside of biological body.

32. The method as claimed in claim 24 wherein said the method of collecting specimen is:
a. Rotation monitoring and control by motorized driving inner core to move inside outside shell rack;
b. Direction monitoring and control with the force interaction between built-in magnet bar and external magnetic field;
c. Sampling and monitoring by two motorized driving blades to open and close;
d. Two samples can be collected with one for each side;
e. Sample storage for each collection;
f. Two blades closing makes a sealing storage space;
g. Pin-hole imager monitors specimen collection.
